(19) United States
(12) Patent Application Publication
(10) Pub. No.: US 2013/0336942 A1
(43) Pub. Date: Dec. 19, 2013

(54) USE OF A PROBIOTIC TO REGULATE BODY WEIGHT

(71) Applicant: CHR-HANSEN A/S, HOERSHOLM (DK)

(72) Inventors: Thomas Dyrmann Leser, Frederiksberg (DK); Thomas GUNNARSSON, Malmoe (SE); Jens KILDSGAARD, Holte (DK); Janni Wandahl PEDERSEN, Alleroed (DK); Bénédicte FLAMBARD, Frederiksberg (DK)

(21) Appl. No.: 13/900,243

(22) Filed: May 22, 2013

Related U.S. Application Data
(62) Division of application No. 13/256,818, filed on Sep. 15, 2011, now abandoned.

(30) Foreign Application Priority Data
Mar. 25, 2009 (EP) 09156109.2

(51) Int. Cl. A61K 35/74; A61K 45/06

(52) U.S. Cl. CPC A61K 35/742; A61K 45/06
USPC 424/93.45

(57) ABSTRACT

The use of probiotic bacteria that modulate expression of a number of satiety markers in the intestine and reduces fat deposition to promote an optimal body weight of a mammal is described. The invention further relates to a composition comprising such a probiotic strain of bacteria and/or a fraction of said strain and/or metabolite of said strain for the preparation of a composition for administration to a mammal for promoting an optimal body weight of a mammal.

### FIELD OF THE INVENTION

The invention relates to the use of probiotic bacteria that modulate expression of satiety markers (e.g. factors coded by the GCG gene) in the intestine while at the same time increasing fat oxidation and reduction of fat deposition in muscle tissue. Consumption of the probiotic strain may thus help to promote an optimal body weight of a mammal. The invention further relates to a composition comprising such a probiotic strain of bacteria and/or a fraction of said strain and/or metabolite of said strain for the preparation of a composition for administration to a mammal for modulating expression of satiety markers in the intestine while at the same time increase fat metabolism in muscle tissue.

### BACKGROUND OF THE INVENTION

### Body Weight Management

The healthy, well functioning body of a mammal (including humans) is characterized by an optimal weight. The specific optimal weight varies widely according to species, gender, age, type of body stature, level of physical activity etc. of the individual mammal. It is however clear that an optimal body weight range can be established for any individual mammal, and that extensive over- as well as under-weight have drastic negative effects on the health and wellbeing of the individual.

In general, during evolution the mammals have adapted to a situation of scarce food resources and famine, and complex mechanisms have evolved to cope with this situation, one being the hunger signaling, which completely changes the behavior pattern of most mammals, and also the mammals' ability to store large energy resources in the form of body fat.

The maintenance of the optimal body weight is complex and multifactorial (NIH 1998). It involves a multitude of signaling pathways and metabolic processes as well as a spectrum of genetic and environmental factors. Present clinical evidence indicates that a multi-faceted intervention involving several signaling pathways and metabolic processes is required to obtain an effect full treatment of obesity.

Within the last decade it has become increasingly clear that the healthy mammalian body also has developed a number of intricate mechanisms that regulate the feed intake during periods of surplus by regulating our satiety. Many of these mechanisms seem to involve specific responses to certain components in the food, and the molecular details of more satiety signaling pathways have been revealed and have shown to involve specific signaling molecules/hormones. Depending on their specific levels (presence or absence) such specific satiety regulating signaling molecules/hormones may signal either satiety or hunger. Collectively they are here referred to as “satiety markers”.

### Satiety Markers

### Proglucagon and Derived Peptide Hormones.

The gene coding for the glucagon precursor, proglucagon, is expressed in the brain, pancreas, and in the small and large intestine. The gene is also referred to as the “GCG gene” or simple “GCG” (Ensembl: ENSG00000115263).”.

In L-cells, located primarily in the epithelium of the distal small intestine and in the colon, the polypeptide proglucagon is cleaved to GLP-1 (glucagon-like peptide 1), GLP-2 (glucagon-like peptide 2), oxyntomodulin, glicentin and IP-2 (intervening peptide 2) (see FIG. 1). While GLP-1 and GLP-2 exert well-defined actions through known receptors, the biological function of glicentin and IP-2 remains less well characterized and no receptors have been identified. The function of oxyntomodulin has been deduced from animal experiments and human trials however, no receptor has yet been identified.

### Physiology of Glucagon-Like Peptide-1 (GLP-1)

In L cells of the intestine, GLP-1 (formerly called insulinotropin) is produced as a 39-amino acid peptide which is stored intracellularly. GLP-1 is released into the circulation in response to nutrient ingestion and has a number of physiological effects (1).

Several studies have demonstrated that the observed improvements in glycemic control following long-term peripheral administration of GLP-1 or pharmacological GLP-1 analogues in animal models and in patients with type 2 diabetes (T2DM) are associated with significant reductions in body weight (2), indicating that GLP-1 may play a role in the regulation of energy balance. In a recent meta-analysis it was concluded that GLP-1 reduces appetite and caloric intake, the latter by an average of 11.7% acutely. The reduction is similar in lean and obese subjects and is achieved without adverse effects (3). It is well-known that GLP-1 inhibits gastric emptying. Reduced gastric emptying generate prolonged stretching of the stomach after food intake. Mechanoreceptors, located in the stomach, quantify the stretch of the stomach and signal satiety to the brain. The satiating effect of GLP-1 may also be caused by GLP-1 acting directly in the brain, as GLP-1 immunoreactive neurons are found in large quantities in the central nerve system (CNS) areas involved in appetite regulation (4-6). Several studies have confirmed the presence of GLP-1 receptors in areas of the brain important to appetite regulation, supporting the notion that GLP-1 is involved in central appetite control (7; 8).

### Physiology of Glucagon-Like Peptide-2 (GLP-2)

As for GLP-1, GLP-2 is produced by posttranslational processing of the polypeptide proglucagon in L cells of the intestine. GLP-2 is as a 33-amino acid peptide which is co-secreted with GLP-1 in response to ingestion of nutrients, especially lipids and carbohydrates (9).

GLP-2 has been proposed to act as a regulator of food intake. When rats received intracerebroventricular administration of GLP-2 food intake was inhibited (10). In contrast to GLP-1, central administration of GLP-2 does not inhibit water intake and does not cause conditioned taste aversion.

In mice, subcutaneous GLP-2 injections enhances intestinal epithelial barrier function affecting both the paracellular and transcellular pathways (11). An improved gastro-intestinal barrier function is associated with a reduction in the flux of lipopolysaccharides (LPS) from the gut lumen into the circulating system. Even moderately increased levels of LPS have recently been shown to induce adipose weight gain similar to what is obtained by a high-fat diet in rodents (12).

### Physiology of Oxyntomodulin

Oxyntomodulin (also referred to as glucagons-37, glicentin-(33-69), and in older references as bioactive enteroglucagon) is produced by posttranslational processing of the polypeptide proglucagon in L cells of the intestine.

Oxyntomodulin shares many properties with GLP-1. Consequently, exogenously administered oxyntomodulin can acutely ameliorate glucose intolerance in diet-induced obese mice and this is likely due to stimulation of glucose-induced insulin secretion (13). Furthermore, oxyntomodulin can delay gastric emptying and reduce gastric acid secretion (14). Importantly, the administration of exogenous oxyntomodulin results in both short-term effects on feeding and more long-term effects on body weight gain in both rodents and human subjects. Oxyntomodulin has been shown to acutely decrease the sensation of hunger and inhibit caloric intake in normal healthy subjects. In the same study, oxyntomodulin administration reduced circulating ghrelin levels by approximately 44% (15). It is possible that the suppressive effect on feeding is mediated via the reduction of ghrelin, a hunger hormone produced by endocrine cells lining the stomach. It has also been suggested that oxyntomodulin may increase energy expenditure (16), which together with reduced energy intake may result in a negative energy balance leading to weight loss. Indeed, seven-day administration (i.p.) of oxyntomodulin reduced the rate of body weight gain and adiposity in rats (17). Similarly, four weeks oxyntomodulin treatment (by subcutaneous injection) resulted in 2.3 kg weight loss (compared to 0.5 kg for the control group) in overweight and obese human subjects (18). These studies clearly indicate that oxyntomodulin may be involved in the regulation of food intake and body weight gain.

### Physiology of Glicentin

Glicentin (also referred to as enteroglucagon, glucagon-69, or gut-type glucagon) corresponds to amino acids 1-69 of preproglucagon. The sequence also comprises the sequence of oxyntomodulin (glicentin-(33-69)). Glicentin 1-30 corresponds to GRPP (glicentin-related pancreatic peptide) (19).

Glicentin is produced in the intestinal L cells and is secreted during digestion. Glicentin slows down gastric emptying and can switch off the duodenojejunal fed motor pattern. Tomita et al. (2005) (20) have reported that glicentin plays an important role in the regulating inhibition of the contraction reaction in normal human jejunum via non-adrenergic non-cholinergic nerves, and has a direct action on the jejunal muscle receptor. In contrast, Ayachi et al. (2005) (21) have shown that glicentin contributes to contraction of smooth muscle cells isolated from human colon. Exendin-(3-39), described as a GLP-1 receptor antagonist, inhibited contraction due to glicentin, suggesting that glicentin may act through the GLP-1 receptor Ayachi et al. (2005) (21).

### The Biology and Physiology of Peptide YY.

Peptide YY (also known as PYY, Peptide Tyrosine Tyrosine, or Pancreatic Peptide YY₃₋₃₆), Ensembl: ENSG00000131096, is encoded by the human chromosome 17 band q21.1. There are two major forms of Peptide YY: PYY₁₋₃₆ and PYY₃₋₃₆. Peptide YY₃₋₃₆ (PYY) is a linear polypeptide consisting of 34 amino acids with structural homology to NPY and pancreatic polypeptide Peptide YY is related to the pancreatic peptide family by having 18 of its 34 amino acids locate in the same positions as pancreatic peptide (22). The most common form of circulating PYY is PYY₃₋₃₆ which binds to the Y2 receptor (Y2R)(23).

PYY is found in L cells in the mucosa of gastrointestinal tract, especially in ileum and colon. A small amount of PYY, about 1-10 percent, is produced in the esophagus, stomach, duodenum and jejunum (24). The plasma PYY concentration increases postprandially (after food ingestion) and decreases by fasting (23).

PYY exerts its action through NPY receptors, inhibits gastric motility and increases water and electrolyte absorption in the colon (25). PYY may also suppress pancreatic secretion. It is secreted by the enteroendocrine cells in the ileum and colon in response to a meal, and has been shown to reduce appetite. PYY works by slowing the gastric emptying; hence, it increases efficiency of digestion and nutrient absorption after a meal.

Several studies have shown that acute peripheral administration of PYY₃₋₃₆ inhibits feeding of rodents and primates. Studies on Y2R-knockout mice have indicated no anorectic (losing appetite) effect on Y2R-knockot mice. These findings suggest that PYY₃₋₃₆ has anorectic effect which is mediated by Y2R. PYY-deficient female mice have increased body weight and fat mass. PYY-deficient mice, on the other hand, are resistant to obesity but have higher fat mass and lower glucose tolerance when fed with high-fat diet, compare to control mice. Thus PYY also plays very important role in energy homeostasis by balancing the food intake (23).

In human volunteers receiving artificial infusions of PYY at normal post-eating concentrations, food intake was reduced by a third for a day. The researchers of this study also investigated the hunger levels of the test group both during and after transfusions of the hormone. The group receiving PYY reported up to a 40% reduction in perceived levels of hunger over a period of 12 hours following infusion (26).

These data suggest the PYY may be useful as a potential therapy for obesity or at least for reducing appetite in individuals on a weight-reduction diet.

### The Biology and Physiology of Apolipoprotein A-IV

Apolipoprotein A-IV is encoded by APOA4 (alias Apo-AIV or ApoA-IV) with gene ID: Ensembl: ENSG00000110244, is encoded by the human chromosome 11 band q23. APOA4 contains 3 exons separated by two introns. The primary translation product is a 396-residue preprotein which after proteolytic processing is secreted in association with chylomicron particles.

Apolipoprotein A-IV (apoA-IV) acts as a satiety factor. ApoA-IV is a 46,000-Da glycoprotein synthesized by the human intestine. In rodents, both the small intestine and liver secrete apoA-IV, but the small intestine is the major organ responsible for circulating apoA-IV (27). There is now solid evidence that the hypothalamus, especially the arcuate nucleus, is another active site of apoA-IV expression (28). Intestinal apoA-IV synthesis is markedly stimulated by fat absorption and does not appear to be mediated by the uptake or re-esterification of fatty acids to form triglycerides. Rather, the local formation of chylomicrons acts as a signal for the induction of intestinal apoA-IV synthesis. Intestinal apoA-IV synthesis is also enhanced by a factor from the ileum, probably peptide tyrosine-tyrosine (PYY)(29). The inhibition of food intake by apoA-IV is mediated centrally (30). The stimulation of intestinal synthesis and secretion of apoA-IV by lipid absorption is rapid; thus apoA-IV likely plays a role in the short-term regulation of food intake. Other evidence suggests that apoA-IV may also be involved in the long-term regulation of food intake and body weight, as it is regulated by both leptin and insulin (28). Chronic ingestion of a high-fat diet blunts the intestinal as well as the hypothalamic apoA-IV response to lipid feeding (29).

Thus, peptide signaling molecules (hormones) such as GLP-1, GLP-2, Oxyntomodulin, Glicentin and PYY as well as proteins such as apolipoprotein A-IV, are involved in the signaling of and the response to either satiety or hunger and can accordingly be referred to as “satiety markers”.

### Fatty Acid Metabolism

### The Biology and Physiology of Stearoyl-CoA Desaturase-1 (SCD1)

Stearoyl-CoA desaturase (SCD; EC 1.14.99.5) is an iron-containing enzyme that catalyzes a rate-limiting step in the synthesis of unsaturated fatty acids. SCD-1 is encoded by a gene on human chromosome 10q24.31 with Entrez gene ID: 6319.

Stearoyl-CoA desaturase-1 (SCD1) determines fatty acid partitioning into lipogenesis or fatty acid β-oxidation in muscle tissue. Upregulation of SCD1 is seen in obese individuals and results in accumulation of intramyocellular triacylglycerol (IMTG). Human obesity is associated with abnormal accumulation of neutral lipids within skeletal myofibers. This phenomenon occurs in concert with reduced insulin stimulated glucose transport and impaired insulin signal transduction. Pharmacological and genetic manipulations that deplete IMTG restore insulin sensitivity. Hulver et al. (2005) (31) have identified a linear relationship between Body Mass Index (BMI) and the expression of SCD1 in muscles in humans. In vitro studies have shown that over-expression of SCD1 in myotubes from lean subjects altered fatty acid partitioning in a manner that resembled the high rates of muscle triacylglycerol (TAG) synthesis and low rates of fatty acid oxidation observed with obesity. The authors proposed “that elevated expression of SCD1 in skeletal muscle may represent a mechanism contributing to reduced fatty acid oxidation, increased IMTG synthesis and progression of the metabolic syndrome”, and further, “that pharmacological targeting of muscle SCD1 and/or its upstream regulators could provide new opportunities for preventing and/or treating obesity and its related co-morbidities (31).

### Probiotics

Probiotic microorganisms have been defined as “Live microorganisms which when administered in adequate amounts confer a health benefit on the host” (FAO/WHO 2001).

It has been described that certain probiotic bacterial strains may have the ability to modulate the level of specific satiety markers.

WO 2007/085970 A2 (DANISCO NS) describes specific strains of Lactobacillus acidophilus, Lactobacillus curvatus, Lactobacillus salivarius and Bifidobacterium lactis that are capable of modulating satiety signaling, specifically mentioning the group of satiety markers consisting of PYY, CCK, Ghrelin, Leptin, GLP-I, orexins, orexigenic hypothalamic neuropeptide Y, acetic acid, amylin and oxyntomodulin.

WO 00238165A1 (PROBI) describes specific Lactobacillus plantarum strains that modulate the levels of insulin and leptin.

US 2004/0048356 A1 (Johansson) describes two specific strains of Lactobacillus plantarum giving rise to increased amounts of propionic acid or acetic acid in the colon, that eventually modify the leptin level and the PPAR gene expression.

However, to the best of our knowledge there is no report of a single class of bacteria that is able to: 1. Modulate all of GLP-1, GLP-2, Oxyntomodulin and Glicentin through activation of the GCG gene in the intestine of an animal, 2. Up-regulate the expression of PYY in the intestine and in cell cultures, 3. Up-regulate the expression of APOA4 in the intestine and 4. Increase fat oxidation on muscle tissue via down-regulation of SCD-1.

### SUMMARY OF THE INVENTION

The present invention relates to a composition comprising at least one strain of Lactobacillus paracasei and/or a fraction of said strain and/or metabolite of said strain for reducing the risk factors involved in overweight and/or obesity, said composition is characterized by up-regulating expression of satiety markers coded by the GCG gene in the intestine of said mammal. In one embodiment said at least one strain and/or a fraction and/or metabolite also up-regulate expression of satiety markers coded by the APOA4 gene and/or satiety markers coded by the Peptide YY in the intestine of the mammal. In a further embodiment, said at least one strain and/or a fraction and/or metabolite in addition down-regulate expression of the SCD1 gene in skeletal muscles of the mammal.

The proglucagon molecule coded by the GCG gene gives rise to a number of known or putative satiety signaling molecules. Accordingly one important aspect of the invention is a Lactobacillus paracasei subsp. paracasei strain and/or a fraction and/or metabolite of said strain which in addition to the modulation of the GCG gene at the mRNA level furthermore modulates the level of one or more of the signaling molecules selected from the group consisting of: GLP-1, GLP-2, Oxyntomodulin, IP-2, GRPP and Glicentin at the protein level. Furthermore, an important aspect of the invention is a Lactobacillus paracasei subsp. paracasei strain and/or a fraction and/or metabolite of said strain which in addition to one or more of the signaling molecules selected from the group consisting of: GLP-1, GLP-2, Oxyntomodulin, IP-2, GRPP and Glicentin, also modulates the level of PYY and/or apolipoprotein A-IV. A further important embodiment of the invention is a Lactobacillus paracasei subsp. paracasei strain and/or a fraction and/or metabolite of said strain which in addition to one or more of the signaling molecules selected from the group consisting of: GLP-1, GLP-2, Oxyntomodulin, IP-2, GRPP, Glicentin, and/or PYY, and/or apolipoprotein A-IV, also down-regulate expression of the SCD1 gene in skeletal muscles of the mammal since the down-modulation of SCD1 is expected to increase fat metabolism and thus augment the efficacy of the Lactobacillus paracasei subsp. paracasei strain (such as CRL431) for reducing overweight and/or treating obesity.

A further aspect of the invention is the use of a composition comprising the strain and/or a fraction and/or metabolite of said strain according to the invention for the preparation of a composition for body weight management of a mammal.

One particularly interesting aspect is the use of a composition comprising the strain and/or a fraction and/or metabolite of said strain according to the invention for the preparation of a medicament for the treatment of overweight or obesity. It is well known that obesity (BMI 30) but also overweight (i.e. BMI 25-30) may have serious medical implications and that both obese and overweight individuals may benefit from a weight reduction. However, even normal or near-normal weight individuals (i.e. BMI 18.5-24.9) who do not suffer under medical implications due to overweight may find it attractive to maintain or strive for an optimal body weight for cosmetic reasons. Thus, one additional aspect of the invention is the use of a composition comprising the strain and/or a fraction and/or metabolite of said strain according to the invention in a cosmetic method for reducing body weight in a non-obese, non-overweight subject having a Body Mass Index (BMI) less than 25, said method comprise providing a composition comprising at least one strain of Lactobacillus paracasei and/or a fraction of said strain and/or metabolite of said strain, wherein said composition is characterized by up-regulating expression of satiety markers coded by the GCG gene and/or the APOA4 gene and/or the Peptide YY gene in the intestine of said mammal. A further embodiment of this aspect is a composition comprising at least one strain of Lactobacillus paracasei and/or a fraction of said strain and/or metabolite of said strain, wherein said composition in addition down-regulate expression of the SCD1 gene in skeletal muscles of the mammal.

The composition comprising the strain and/or a fraction and/or metabolite of said strain according to the invention may be formulated in both liquid and solid dosage forms. In the latter case, the product may be powdered and formed into tablets, granules or capsules or simply mixed with other food ingredients to form a functional food. Accordingly in one aspect the composition comprising the strain and/or a fraction and/or metabolite of said strain according to the invention is used for the preparation of a functional food or feed intended to control or stabilize the weight gain of a mammal.

### DEFINITIONS

Prior to a discussion of the detailed embodiments of the invention is provided a definition of specific terms related to the main aspects of the invention.

As used herein the term “BMI” designates body mass index. BMI is a measure of the weight of a person scaled according to height. It is defined as the individual's body weight divided by the square of their height (weight measured in kilograms, height in meters). The formula universally used in medicine produce a unit of measure of kg/m². According to the US Department of Health & Human Services a BMI below 18.5 indicates underweight, 18.5-24.9 normal weight, 25-29.9 overweight and a BMI of 30 and above indicates obesity. It should be noted that not only obesity but also overweight (BMI 25-29.9) increases the risk of mortality in adults (Neovius et al (2009) BMJ 338, b496). Accordingly overweight is not only of relevance because of cosmetic indications but also for its medical implications.

By the expression “risk factors involved in overweight and/or obesity” is referred to one or more of the many biochemical factors that are negatively involved in the development of overweight and/obesity. One particular interesting group of such risk factors are the so-called “satiety markers”. By the term “satiety markers” is referred to peptides or hormones (including the genes coding for said peptides/hormones) that are involved in the signaling of and the response to satiety which upon increased levels of the factors provide an animal with the feeling of satiety and thereby lead to a reduced feed intake. GLP-1, GLP-2, Oxyntomodulin, and Glicentin, and PYY are examples of such peptide signaling molecules.

The gene referred to as the “GCG gene” or simply “GCG” is the gene coding for the glucagon precursor, proglucagon, the GCG gene is also referred to as the “proglucagon gene”.

By the expression “probiotics or probioticum” is referred to a composition which comprises probiotic microorganisms. Probiotic bacteria are defined as microbial cells that have a beneficial effect on the health and well-being of the host. Probiotic microorganisms have been defined as “Live microorganisms which when administered in adequate amounts confer a health benefit on the host” (FAO/WHO 2001).

By the expression “prebiotic” is referred to a composition or a component of a composition which increases the number of probiotic bacteria in the intestine. Thus, prebiotics refer to any non-viable food component that is specifically fermented in the colon by indigenous bacteria thought to be of positive value, e.g. bifidobacteria, lactobacilli. The combined administration of a probiotic strain with one or more prebiotic compounds may enhance the growth of the administered probiotic in vivo resulting in a more pronounced health benefit, and is termed synbiotic.

Embodiments of the present invention are described below, by way of examples only.

### DETAILED DISCLOSURE OF THE INVENTION

The invention relates to the use of probiotic bacteria to promote an optimal body weight of a mammal. To the surprise of the inventors, compositions comprising certain live probiotic bacteria, in particular Lactobacillus paracasei subsp. paracasei strain CRL431 bacteria are able specifically to induce the expression of: 1. The GCG gene in the distal ileum part of the intestine of a mammal (example 1), 2. The APOA4 gene (example 4), 3. GLP1, GLP-2 and PYY secretion in the distal ileum part of the intestine of a mammal (example 2), 4. Secretion of PYY from a human enteroendocrine cell line (example 3), and 5. Decreased expression of SCD-1 in skeletal muscle tissue (example 5). Without wishing to be bound by theory it is perceivable that not only living Lactobacillus paracasei subsp. paracasei cells but also a fraction of said bacteria or even a metabolite of said strain can be used for the preparation of a composition for administration to a mammal for modulating expression of the GCG and APOA4 genes gene in the intestine, increasing PYY secretion in the intestine, and increasing fat oxidation in skeletal muscle tissue.

Although the invention is not to be construed as limited by any particular theory, it is thought that due to the fact that maintenance of the optimal body weight involves a multitude of individual signaling pathways and metabolic processes, it follows that a Lactobacillus paracasei subsp. paracasei strain which up-regulates more satiety-factors will prove to have improved efficacy.

Accordingly in one embodiment of the invention the strain and/or a fraction and/or metabolite of said strain is used to induce an increased expression of the GCG gene and also the APOA4 and/or the Peptide YY gene in the intestine. In a further embodiment of the invention the invention the strain and/or a fraction and/or metabolite of said strain is used to induce a reduced expression of the SCD1 gene in skeletal muscles of the mammal.

As illustrated in example 1 the probiotic Lactobacillus paracasei subsp. paracasei strain CRL431 (ATCC 55544) is particularly effective in activating the expression of the GCG gene.

As further illustrated in example 2-5 the probiotic Lactobacillus paracasei subsp. paracasei strain CRL431 (ATCC 55544) also up-regulates expression of the APOA4 gene and the PYY gene, in the intestine, and furthermore down-regulate expression of the SCD1 gene in skeletal muscles of said mammal. Thus this Lactobacillus paracasei subsp. paracasei strain appears to be unique in that it up-regulates a multitude of satiety-factors and in addition increase fatty acid oxidation in the muscles.

It is contemplated that strains that are directly derived from this probiotic strain are likely to retain their probiotic features including the feature of increasing the expression of genes coding for various satiety-factors and/or lipid metabolism.

Accordingly, one preferred embodiment of the invention is the use of Lactobacillus paracasei subsp. paracasei strain and/or a fraction of said strain and/or metabolite of said strain for reducing the risk factors involved in overweight and/or obesity, wherein the strain is selected from the group of strains consisting of Lactobacillus paracasei subsp. paracasei (CHCC3136, CRL431, ATCC 55544) and a mutant strain thereof, wherein the mutant strain is obtained by using ATCC 55544 as starting material, and wherein the mutant has retained or further improved the ability to up-regulate expression of the GCG gene and/or the APOA4 gene and/or the PYY gene, in the intestine, and/or has retained or further improved the ability to down-regulate expression of the SCD1 gene in skeletal muscles of said mammal.

The strain Lactobacillus paracasei subsp. paracasei CRL431 was deposited according to the Budapest Treaty on the International Recognition of the Deposit of Microorganisms for the Purposes of Patent Procedure with the American Tissue type Collection Center, 10801 University Blvd, Manassas, Va. 20110, USA on 24 Jan. 1994 under accession number ATCC 55544. The CRL431 strain is commercially available from Chr. Hansen A/S, 10-12 Boege Alle, DK-2970 Hoersholm, Denmark, under the product name Probio-Tec® F-DVS L. casei-431®, Item number 501749, and under the product name Probio-Tec® C-Powder-30, Item number 687018.

In the intestine the proglucagon mRNA gives rise to the production of a number of peptides [ex. 2 expression of biological active protein GLP-1 GLP-2 and PYY increased] also, i.e. GLP-1, GLP-2, Oxyntomodulin, IP-2, GRPP and Glicentin, most of which are known to be or suspected to be signaling molecules involved the signaling of and the response to either satiety or hunger

To further substantiate the finding that CRL431 increase the expression of the GCG gene, the gene products, i.e. intact GLP-1, GLP-2, Oxyntomodulin and total GLP-1 as well as GLP-2, were measured in the venous effluent of an isolated pig intestine perfused with the CRL431 strain (see example 2). As shown in FIG. 3, the concentration of intact, biologically active GLP-1 increased by 443% following perfusion of the isolated pig intestine with CRL431. The levels of total GLP-1 and GLP-2 increase by 330% and 460%, respectively, indicating an increased secretion of GCG derived proglucagon hormones. Furthermore, secretion of the satiety inducing hormone PYY was also induced by perfusion with CRL431. As indicated in FIG. 3, PYY levels increased by 228% after perfusion with CRL431. Therefore one preferred embodiment of the invention is the use of at least one strain of bacteria and/or a fraction of said strain and/or metabolite of said strain of the invention for the preparation of a composition for administration to a mammal for modulating expression of one or more of the signaling molecules selected from the group consisting of: GLP-1, GLP-2, Oxyntomodulin, IP-2, GRPP and Glicentin.

As further illustrated in example 3, CRL431 induces the secretion of PYY in cell cultures of a human enteroendocrine cell line (NCI-H716). In this example, CRL431 induce PYY secretion in a dose-dependent manner to reach levels 1131% above background.

Evidence clearly indicates that GLP-1, GLP-2, Oxyntomodulin, Glicentin, and PYY are hormones that are either directly or indirectly (e.g. via increased intestinal epithelial barrier function) able to promote an optimal body weight of a mammal. Accordingly a further embodiment is the use of at least one strain of bacteria and/or a fraction of said strain and/or metabolite of said strain for the preparation of a composition for the preparation of a cornposition for body weight management. In particularly the use of at least one strain of bacteria and/or a fraction of said strain and/or metabolite of said strain for the preparation of a medicament for the treatment of overweight and/or obesity is a preferred embodiment of the invention.

In example 4, we show that the probiotic Lactobacillus paracasei subsp. paracasei strain CRL431 (ATCC 55544) increase the expression of the APOA4 gene in intestinal tissue. It has been shown that the APOA4 gene product, apolipoprotein A-IV is a satiety inducing protein. Accordingly, one embodiment of the invention is the use of at least one strain of Lacto-bacillus paracasei subsp. paracasei and/or a fraction of said strain and/or metabolite of said strain for the preparation of a composition for administration to a mammal for modulating expression of the APOA4 gene in intestinal tissue in said mammal

Furthermore, as illustrated in example 5 the probiotic Lactobacillus paracasei subsp. para-casei strain CRL431 (ATCC 55544) decrease the expression of the SCD-1 gene in skeletal muscle tissue. It is contemplated that strains that are directly derived from this probiotic strain are likely to retain their probiotic features including the feature of decreasing the expression of the SCD-1 gene in skeletal muscle tissue. Accordingly, one embodiment of the invention is the use of at least one strain of bacteria and/or a fraction of said strain and/or metabolite of said strain for the preparation of a composition for administration to a mammal for modulating expression of the SCD-1 gene in the muscle tissue in said mammal. A further preferred embodiment is the use of at least one strain of Lactobacillus paracasei wherein the at least one strain and/or a fraction and/or metabolite up-regulate expression of satiety markers coded by the GCG, the APOA4 and the Peptide YY gene in the intestine of the mammal and also down-regulate expression of the SCD1 gene in skeletal muscles of the mammal. A particularly preferred embodiment is the use of at least one strain of Lactobacillus paracasei subsp. paracasei wherein the at least one strain and/or a fraction and/or metabolite of said strain is selected from the group of strains consisting of Lactobacillus paracasei subsp. paracasei strain CRL431 (ATCC 55544) and a mutant strain thereof, wherein the mutant strain is obtained by using ATCC 55544 as starting material and wherein the mutant has retained or further improved the ability to up-regulate expression of the GCG gene, the APOA4 gene or the PYY gene, in the intestine, or has retained or further improved the ability to down-regulate expression of the SCD1 gene in skeletal muscles of said mammal.

Obesity is a major risk factor for developing a number of diseases and symptoms. According to The Endocrine Society or The Hormone Foundation (http://www.obesityinamerica.org) overweight and obese people are at an increased risk for developing the following conditions: cardiovascular diseases (e.g. atherosclerosis, hypertension, stroke, congestive heart failure, Angina pectoris), type 2 diabetes mellitus, obesity-related hypoventilation, back and joint problems, non-alcoholic fatty liver disease, gastroesophageal reflux disease, reduced fertility, hypothyroidism, dyslipidemia, hyperinsulinemia, cholecystitis, cholelithiasis, osteoarthritis, gout, sleep apnea and other respiratory problems, polycystic ovary syndrome (PCOS), pregnancy complications, psychological disorders, uric acid nephrolithiasis (kidney stones), stress urinary incontinence and increased incidence of certain cancers (e.g. cancer of the kidney, endometrium, breast, colon and rectum, esophagus, prostate and gall bladder).

Type 1 diabetes (T1 DM) is the result of autoimmune destruction of the insulin-producing β-cells in the pancreas. Animal studies indicate that GLP-1 therapy may delay the onset of T1 DM by inducing β-cell neogenesis and proliferation (32)22). It has been suggested that this effect is related to anti-inflammatory properties of GLP-1 (33, 23).

Accordingly, yet an embodiment of the invention is the use of the strain and/or a fraction and/or metabolite of said strain (such as CRL431) of the invention for the preparation of a composition or medicament for the prevention and/or treatment of anyone of the above mentioned diseases or conditions.

Many probiotics are used for the manufacture of food or feed products; consequently a further important aspect of the invention is the provision of a human or animal food or feed composition comprising the Lactobacillus paracasei subsp. paracasei strain and/or a fraction and/or metabolite of said strain of the invention to control or stabilize the weight gain of a mammal. Such food or feed are frequently referred to as functional food or feed.

When preparing such food or feed products manufacturers usually make use of a so-called starter cultures being cultures used to process food and feed products. Starter cultures are widely used in the diary industry. Typically starter cultures impart specific features to various food or feed products. It is a well established fact that the consistency, texture, body and mouth feel is strongly related to the EPS production of the starter culture used to prepare the food or feed.

The present invention also devices a method of manufacturing a food or feed product comprising adding a starter culture composition comprising Lactobacillus paracasei subsp. paracasei CRL431 (ATCC 55544) or a mutant strain thereof to a food or feed product starting material and keeping the thus inoculated starting material under conditions where the lactic acid bacterium is metabolically active to obtain a food or feed product to control or stabilize the weight gain of a mammal.

By the expression “prebiotic” is referred to a composition or a component of a composition which increases the number of probiotic bacteria in the intestine. Thus, prebiotics refer to any non-viable food component that is specifically fermented in the colon by indigenous bacteria thought to be of positive value, e.g. lactobacilli. The combined administration of a probiotic strain with one or more prebiotic compounds may enhance the growth of the administered probiotic in vivo resulting in a more pronounced health benefit. Therefore one further embodiment of the invention is the use of a composition comprising living probiotic bacteria according to the invention in combination with at least one prebiotic. An embodiment wherein the prebiotic is selected from the group: inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides) and polydextrose is especially preferred.

### THE INVENTION PRESENTED IN THE FORM OF CLAIMS

Preferred aspects and embodiments of the invention may be presented in the form of so-called claims. These are given below.

1. A composition comprising at least one strain of Lactobacillus paracasei subsp. paracasei and/or a fraction of said strain and/or metabolite of said strain for reducing the risk factors involved in overweight and/or obesity, said composition is characterized by up-regulating expression of satiety markers coded by the GCG gene in the intestine of said mammal.

2. The composition according to claim 1, wherein the up-regulation of expression of the GCG gene occur in the distal ileum part of the intestine of a mammal.

3. The composition according to any of the preceding claims, wherein the at least one strain and/or a fraction and/or metabolite also up-regulate expression of satiety markers coded by the APOA4 gene in the intestine of the mammal.

4. The composition according to any of the preceding claims, wherein the at least one strain and/or a fraction and/or metabolite also increase secretion of the satiety marker Peptide YY in the intestine of the mammal.

5. The composition according to any of the preceding claims, wherein the at least one strain and/or a fraction and/or metabolite also down-regulate expression of the SCD1 gene in skeletal muscles of the mammal.

6. The composition according to any of the preceding claims, wherein the at least one strain and/or a fraction and/or metabolite increase PYY secretion from the intestine, up-regulate expression of satiety markers coded by the GCG, and the APOA4 genes in the intestine of the mammal and also down-regulate expression of the SCD1 gene in skeletal muscles of the mammal.

7. The composition according to any of the preceding claims, wherein the strain is selected from the group of strains consisting of Lactobacillus paracasei subsp. paracasei (CHCC3136, CRL431, ATCC 55544) and a mutant strain thereof, wherein the mutant strain is obtained by using ATCC 55544 as starting material, and wherein the mutant has retained or further improved the ability to up-regulate expression of the GCG gene or the APOA4 gene or further improved the ability to increase PYY secretion from the intestine, or has retained or further improved the ability to down-regulate expression of the SCD1 gene in skeletal muscles of said mammal.

8. The composition according to any of the preceding claims, wherein the strain and/or a fraction and/or metabolite of said strain furthermore modulate the level of one or more of the signaling molecules selected from the group consisting of: GLP-1, GLP-2, Oxyntomodulin, IP-2, GRPP, Glicentin, PYY and Apolipoprotein A-IV.

9. The composition according to any of the preceding claims for the prevention and/or treatment of a disease or condition selected from the group of obesity and obesity-related diseases consisting of cardiovascular diseases (e.g. atherosclerosis, hypertension, stroke, congestive heart failure, Angina pectoris), type 1 diabetes mellitus, type 2 diabetes mellitus, obesity-related hypoventilation, back and joint problems, non-alcoholic fatty liver disease, gastroesophageal reflux disease, reduced fertility, hypothyroidism, dyslipidemia, hyperinsulinemia, cholecystitis, cholelithiasis, osteoarthritis, gout, sleep apnea and other respiratory problems, polycystic ovary syndrome (PCOS), pregnancy complications, psychological disorders, uric acid nephrolithiasis (kidney stones), stress urinary incontinence and certain cancers (e.g. cancer of the kidney, endometrium, breast, colon and rectum, esophagus, prostate and gall bladder).

10. A cosmetic method for reducing body weight in a non-obese, non-overweight subject having a Body Mass Index (BMI) less than 25, said method comprise providing a composition comprising at least one strain of Lactobacillus paracasei subsp. paracasei and/or a fraction of said strain and/or metabolite of said strain, wherein said composition is characterized by up-regulating expression of satiety markers coded by the GCG gene in the intestine of said mammal.

11. A cosmetic method for reducing body weight in a non-obese subject, said method cornprise providing a composition comprising at least one strain of Lactobacillus paracasei subsp. paracasei and/or a fraction of said strain and/or metabolite of said strain, wherein said composition is characterized by up-regulating expression of satiety markers coded by the GCG gene in the intestine of said mammal.

12. The cosmetic method according to any of claim 10 or 11, wherein the at least one strain and/or a fraction and/or metabolite also up-regulate expression of satiety markers coded by the APOA4 gene in the intestine of the mammal.

13. The cosmetic method according to any of claims 10 to 12, wherein the at least one strain and/or a fraction and/or metabolite also up-regulate secretion of the satiety marker Peptide YY in the intestine of the mammal.

14. The cosmetic method according to any of claims 10 to 13, wherein the at least one strain and/or a fraction and/or metabolite also down-regulate expression of the SCD1 gene in skeletal muscles of the mammal.

15. The cosmetic method according to any of claims 10 to 14, wherein the at least one strain and/or a fraction and/or metabolite increase PYY secretion from the intestine, up-regulate expression of the GCG and the APOA4 gene in the intestine, and also down-regulate expression of the SCD1 gene in skeletal muscles of said mammal

16. The cosmetic method according to any of claims 10 to 14, wherein the strain is selected from the group of strains consisting of Lactobacillus paracasei subsp. paracasei (CHCC3136, CRL431, ATCC 55544) and a mutant strain thereof, wherein the mutant strain is obtained by using ATCC 55544 as starting material, and wherein the mutant has retained or further improved the ability to up-regulate expression of the GCG gene, the APOA4 gene or the secretion of PYY in the intestine, or has retained or further improved the ability to down-regulate expression of the SCD1 gene in skeletal muscles of said mammal.

17. The composition according to any of the preceding claims, wherein the at least one strain and/or a fraction and/or metabolite is used for the preparation of a food or feed intended to control or stabilize the weight gain of a mammal.

18. The composition according to any of the preceding claims, wherein the at least one strain and/or a fraction and/or metabolite is combined with at least one prebiotic.

19. A composition according to claim 18, wherein the at least one strain and/or a fraction and/or metabolite is combined with at least one prebiotic, wherein the at least one prebiotic is selected from the group consisting of: inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides), and polydextrose.

20. The use of at least one strain of Lactobacillus paracasei subsp. paracasei and/or a fraction of said strain and/or metabolite of said strain for the preparation of a medicament for administration to a mammal for reducing the risk factors involved in overweight and/or obesity, including up-regulating expression of satiety markers coded by the GCG gene in the intestine of said mammal.

21. The use according to claim 20, wherein the up-regulation of expression of the GCG gene occur in the distal ileum part of the intestine of a mammal.

22. The use according to claim 20 or 21, wherein the at least one strain and/or a fraction and/or metabolite also up-regulate expression of satiety markers coded by the APOA4 gene in the intestine of the mammal.

23. The use according to claims 20 to 22, wherein the at least one strain and/or a fraction and/or metabolite also up-regulate secretion of the satiety markers coded by the Peptide YY gene in the intestine of the mammal.

24. The use according to claims 20 to 23, wherein the at least one strain and/or a fraction and/or metabolite also down-regulate expression of the SCD1 gene in skeletal muscles of the mammal.

25. The use according to claims 20 to 24, wherein the at least one strain and/or a fraction and/or metabolite up-regulate expression of satiety markers coded by the GCG, the APOA4 increase PYY secretion in the intestine of the mammal and also down-regulate expression of the SCD1 gene in skeletal muscles of the mammal.

26. The use according to claims 20 to 25, wherein the strain is selected from the group of strains consisting of Lactobacillus paracasei subsp. paracasei (CHCC3136, CRL431, ATCC 55544) and a mutant strain thereof, wherein the mutant strain is obtained by using ATCC 55544 as starting material, and wherein the mutant has retained or further improved the ability to up-regulate expression of the GCG gene, the APOA4 gene or the PYY secretion, in the intestine, or has retained or further improved the ability to down-regulate expression of the SCD1 gene in skeletal muscles of said mammal.

27. The use according to claims 20 to 26, wherein the strain and/or a fraction and/or metabolite of said strain according to any of the preceding claims furthermore modulate the level of one or more of the signaling molecules selected from the group consisting of: GLP-1, GLP-2, Oxyntomodulin, IP-2, GRPP, Glicentin, PYY and Apolipoprotein A-IV.

The invention is further illustrated in the following non-limiting examples and the figures.

### BRIEF DESCRIPTION OF THE DRAWINGS

FIG. 1: Schematic representation of the human proglucagon molecule and the proglucagon-derived peptides produced in L-cells of the small and large intestine. The numbers refer to the relative amino acid positions within proglucagon. GRPP: Glicentin-related pancreatic polypeptide. Figure adapted from Wallis et al. 2007 (19).

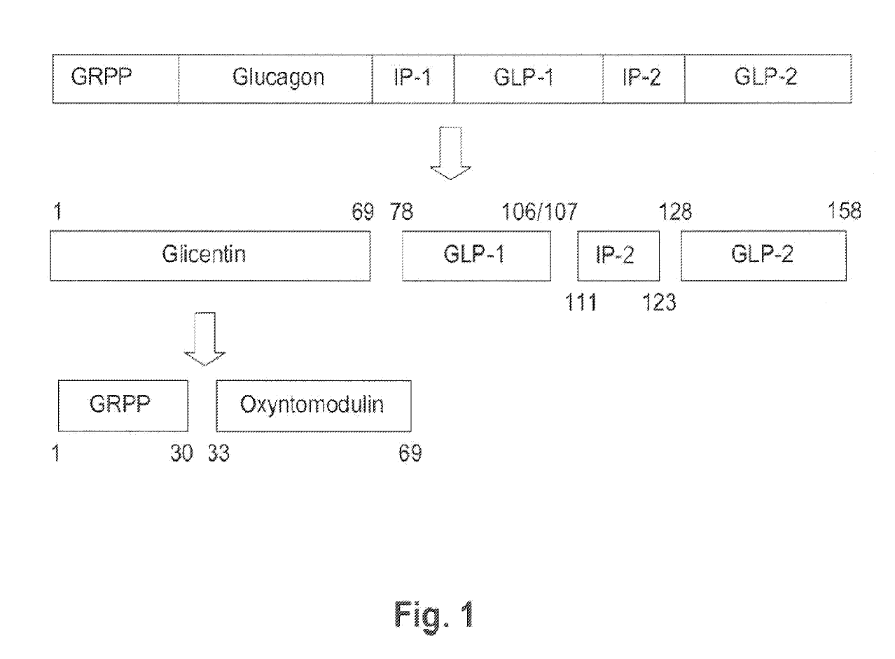

FIG. 2: Expression of GCG in porcine ileum. GCG expression was quantified by Q-PCR on RNA extracted from mucosa samples. Each dot represents an individual pig. Average expression is presented as a black line. The average expression of the control group (crtl) was set at 1.0. Bb12: Bifidobacterium animalis subsp. lactis strain BB-12® (DSM15954); La5: Lactobacillus acidophilus strain La5 (DSM13241); CRL431: Lactobacillus paracasei subsp. paracasei strain CRL431, (ATCC 55544). *** p<0.001 (t-test).

FIG. 3: The central ileum of an overnight fasted pig was isolated. The artery, vein, and gut lumen of the isolated ileum were perfused with buffers as described. The perfusion was carried out from time point 0 to 220 minutes. The isolated ileum was stimulated six times with arterial bombesin (indicated as vertical dashed lines). The gut lumen was perfused with CRL431 from time points 39 to 146 minutes (indicated by the horizontal capped line). The venous effluent was collected at 1 minute periods. Intact and total GLP-1, GLP-2, and somatostatin was analyzed in the venous effluent using previously described radioimmunoassays and PYY was analyzed in the venous effluent using ELISA kits from Linco (Millipore) (34).

FIG. 4: Release of PYY from NCI-H716 enteroendocrine cells stimulated with CRL431 for two hours. NCI-H716 enteroendocrine cells were cultivated as described and CRL-431 coincubated with the cell culture at indicated concentrations.

FIG. 5. Expression of APOA4 in intestinal tissue. SCD-1 expression was quantified by Q-PCR on RNA extracted from mucosa samples. Each column represent average expression values and the error bar represent standard deviation. The average expression value of the control group (crtl) was set at 1.0 (n=6 piglets). Bb12: Bifidobacterium animalis subsp. lactis strain BB-12® (DSM15954) (n=7 piglets); La5: Lactobacillus acidophilus strain La5 (DSM13241) (n=6 piglets); CRL431: Lactobacillus paracasei subsp. paracasei strain CRL431, (ATCC 55544) (n=5 piglets).

FIG. 6. Expression of SCD-1 in skeletal muscle. SCD-1 expression was quantified by Q-PCR on RNA extracted from mucosa samples. Each column represent average expression values and the error bar represent standard deviation. The average expression value of the control group (crtl) was set at 1.0 (n=6 piglets). Bb12: Bifidobacterium animalis subsp. lactis strain BB-12® (DSM15954) (n=7 piglets); La5: Lactobacillus acidophilus strain La5 (DSM13241) (n=6 piglets); CRL431: Lactobacillus paracasei subsp. paracasei strain CRL431, (ATCC 55544) (n=5 piglets).

FIG. 7. Ad libitum energy intake four hours after intake of capsule A (placebo), B (10⁹ CFU), or C (10¹⁹ CFU). N=21, mean±SE.

### EXAMPLES

### Example 1

### Probiotic Strains Up-Regulate GCG Expression in the Ileum of Pigs

To investigate whether or not selected probiotic strains regulate ileal GCG expression in animals, young pigs were fed a standard diet including probiotic bacteria (i.e. Bifidobacterium animalis subsp. lactis strain BB-12® (DSM15954), Lactobacillus acidophilus strain La5 (DSM13241), and Lactobacillus paracasei subsp. paracasei strain CRL431, (ATCC 55544). The BB-12® strain is commercially available from Chr. Hansen A/S, 10-12 Boege Alle, DK-2970 Hoersholm, Denmark. Pigs fed with the same standard diet but not supplemented with probiotic bacteria served as control. Each group consisted of 8 piglets. At weaning at 4 weeks the animals were moved to pens where they were housed individually and assigned to the corresponding treatments for 14 days. Littermates were assigned to each of the treatments. The number of barrows and gilts in each treatment was the same. The pigs were fed twice daily, receiving an amount of feed corresponding to 4% of their body weight. The probiotics were given on top of the diet every morning.

Permission to carry out the experiment was granted from The Danish Plant Directorate and The Danish Ministry of Food, Agriculture and Fisheries.

After 14 days of treatment, the pigs were killed and tissues comprising 75% of the full length of the small intestine (i.e. the ileum or distal part of the small intestine) were sampled and snap-frozen in liquid nitrogen. Gene expression analysis on the distal ileum was performed by quantitative PCR analysis using primers specific for GCG. The quantitative PCR analysis was performed essentially as described by Kubista et al. (3524).

Primer sequences were,

GCG-F: 5′-TTC AGA ATA CAG AGG AGA AAT CCA-3′
GCG-R: 5′-AGT CAT CTG ATC TGG ATC ATC G-3′

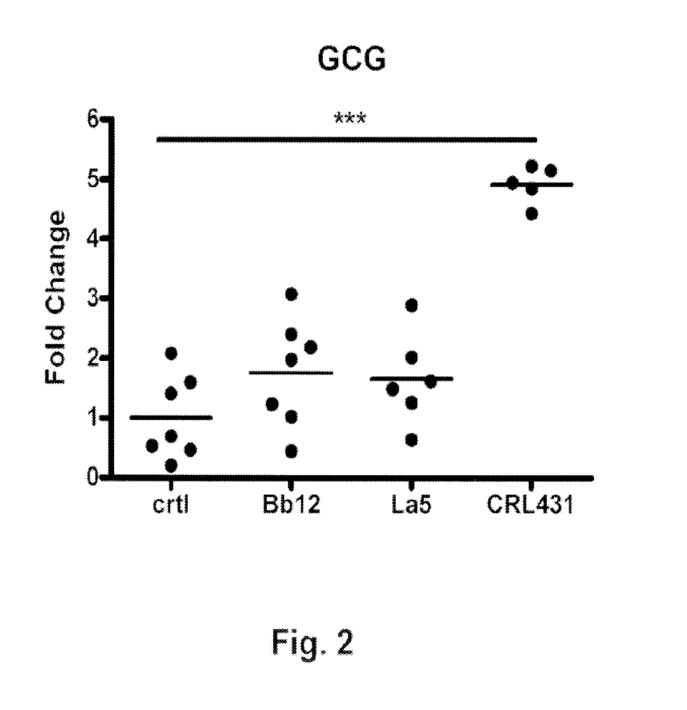

As indicated in FIG. 2, CRL-431 significantly up-regulates ileal GCG expression while the two other strains had no significant effect on GCG expression.

### Example 2

### Effect of CRL431 on Gut Hormone Release in Isolated Perfused Pig Intestine

One overnight fasted Danish LYY strain pig (XX kg) was anesthetized and an 80 cm section of the central ileum, including arterial and venous supply, was isolated. The segment was perfused with gassed (5% CO₂ in O₂) Krebs-Ringer bicarbonate perfusion buffer containing 0.1% human serum albumin; 5% dextran T-70; 7 mmol/L glucose; a mixture of amino acids (5 mmol/L); and 15-20% freshly washed bovine erythrocytes. A cyclooxygenase inhibitor was added to prevent generation of prostaglandins in the perfusion system. The tissue was perfused with the buffer at 24 mL/min. The gut lumen was perfused with perfusion buffer without erythrocytes at 3 mL/min. Perfusion pressure was recorded continuously and oxygen status and glucose levels were analyzed approx. every 30 min in venous effluent. The venous effluent was collected for 1 min periods. After centrifugation at 4° C., the supernatants were divided into appropriate aliquots and stored in the freezer until analysis.

The experimental protocol consisted of perfusion of gut lumen with CRL431 (10⁸ CFU/mL) alone or in combination with an arterial bombesin stimuli (10⁻⁸ M).

Intact and total GLP-1, GLP-2, oxyntomodulin, and somatostatin were measured using previously described radioimmunoassays (14; 34)25). PYY was measured using an ELISA kit from Linco (Millipore, Mass., USA).

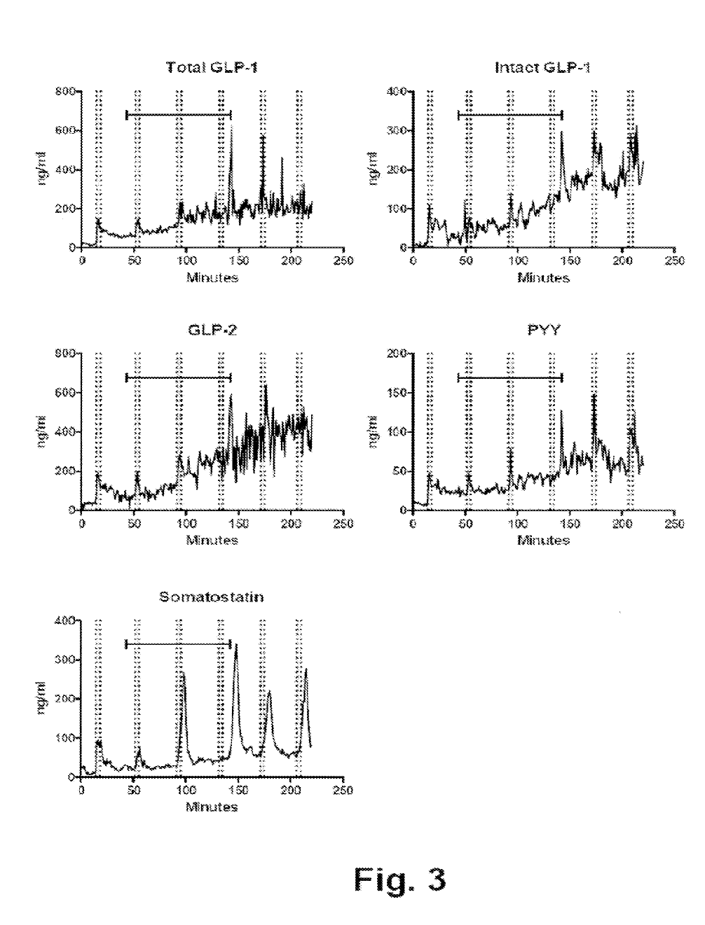

As indicated in FIG. 3, background levels of total GLP-1 secretion are at 75 ng/ml. Background levels of the released hormones are defined as the average of the measurements after the first bombesin stimulus, i.e. measurements obtained at time points 19-33 minutes. Perfusion of the intestinal lumen with CRL431 was carried out from time point 34 to 141 minutes. Perfusion with CRL431 gradually increases total GLP-1 levels to 215 ng/ml (defined as the average of the measurements after the first bombesin stimulus after bacterial infusion, i.e. measurements obtained at time points 176-205 minutes). The measurements correspond to a 287% increase in total GLP-1 levels.

An increase in the levels of intact, biologically active GLP-1 was also observed. Perfusion of the isolated pig intestine increased the levels of intact GLP-1 from 54 to 182 ng/ml, corresponding to an increase of 339%. Similarly, GLP-2 levels increased from 105 to 405 ng/ml, corresponding to an increase of 383%.

It is well-known that somatostatin inhibit the secretion of GLP-1 (36). In order to verify that the increased levels of intact and total GLP-1 and GLP-2 are not a result of decreased somatostatin expression from intestinal L-cells, we measured the levels of this hormone in the venous effluent. As shown in FIG. 3, the concentration of somatostatin increases during CRL431 perfusion from 40 to 93 ng/ml, corresponding to an increase of 237%. Thus, the increased expression of the proglucagon-derived hormones GLP-1 and GLP-2 is not due to a decrease in the expression of somatostatin.

Furthermore, as indicated in FIG. 3, background levels of PYY secretion are at 28 ng/ml. Perfusion with CRL431 gradually increases PYY levels to 65 ng/ml. The measurements correspond to a 228% increase in plasma PYY levels after CRL431 perfusion.

A surprising and highly interesting observation is seen after perfusion of the intestine with the Lactobacillus paracasei subsp. paracasei strain CRL431. After bacterial perfusion, the bombesin-induced release of total and intact GLP-1, GLP-2 and PYY is intensified with peak values of 624, 298, 639, and 148 ng/ml, respectively. This should be compared to peak values of total and intact GLP-1, GLP-2 and PYY of 281, 139, 316 and 79 ng/ml, respectively, during bacterial infusion.

In summary we have shown that CRL431 increase the levels of secreted GLP-1, GLP-2, and PYY, and that the bombesin-induced release GLP-1 GLP-2, and PYY is intensified (peak values).

### Example 3

### Effect of CRL431 on PYY Release in Cultured Human Enteroendocrine Cells

Human enteroendocrine cells, NCI-H716, were cultured as previously described (37). CRL431 was cultivated in MRS (deMan, Rogosa and Sharpe, Difco, BD Diagnostics) and allowed to reach exponential growth phase. The bacteria were harvested by centrifugation and resuspeded after a single wash step in Krebs-Ringer buffer (Sigma, K4002) with 0.2% BSA (Sigma, A3294) to obtain the following concentrations: 1×10⁷, 5×10⁷, 7.5×10⁷ and 1×10⁸ cfu/ml.

Bacteria were incubated with the cell culture for two hours at 37° C. After incubation the supernatants were collected in test tubes containing dipeptidyl peptidase-4 (DPP-4) inhibitor (Millipore, Mass., USA). PYY concentration of the cell culture supernatants was determined by ELISA (Linco, Millipore, Mass., USA).

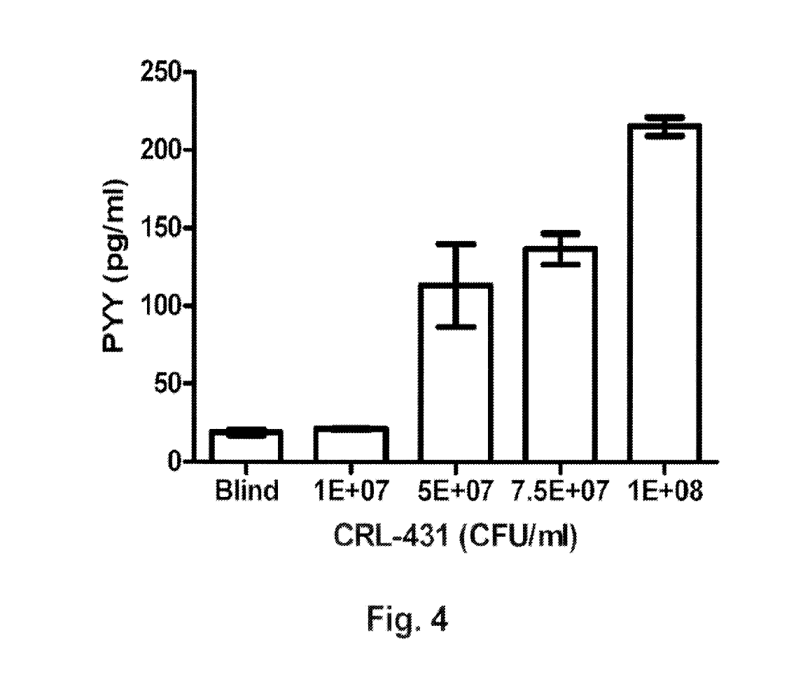

As indicated in FIG. 4 background levels of PYY were 19 pg/ml. Incubation of the NCI-H716 cell culture with increasing concentration of CRL431 triggers a dose-dependent release of PYY. At the highest concentration of CRL431, the PYY concentration reached a maximum of 215 pg/ml corresponding to an increase of 1131%.

### Example 4

### Probiotic Strain Upregulate APOA4 Expression in the Pig Intestine

To investigate whether or not selected probiotic strains regulate ileal APOA4 expression in animals, young pigs were fed a standard diet including probiotic bacteria (i.e. Bifidobacterium animalis subsp. lactis strain BB-12® (DSM15954), Lactobacillus acidophilus strain La5 (DSM13241), and Lactobacillus paracasei subsp. paracasei strain CRL431, (ATCC 55544). Pigs fed with the same standard diet but not supplemented with probiotic bacteria served as control. Each group consisted of 8 piglets. At weaning at 4 weeks the animals were moved to pens where they were housed individually and assigned to the corresponding treatments for 14 days. Littermates were assigned to each of the treatments. The number of barrows and gilts in each treatment was the same. The pigs were fed twice daily, receiving an amount of feed corresponding to 4% of their body weight. The probiotics were given on top of the diet every morning.

Permission to carry out the experiment was granted from The Danish Plant Directorate and the Danish Ministry of Food, Agriculture and Fisheries.

After 14 days of treatment, the pigs were killed and tissues comprising 75% of the full length of the small intestine (i.e. the ileum or distal part of the small intestine) were sampled and snap-frozen in liquid nitrogen. Gene expression analysis on the distal ileum was performed by quantitative PCR analysis using primers specific for APOA4. The quantitative PCR analysis was performed essentially as described by Kubista et al. (35).

Primer sequences were,

APOA4-F: 5′- AAGGCCAAGATCGATCAGAA -3′
APOA4-R: 5′- GAGCTCCTCCGCATAGGG -3′

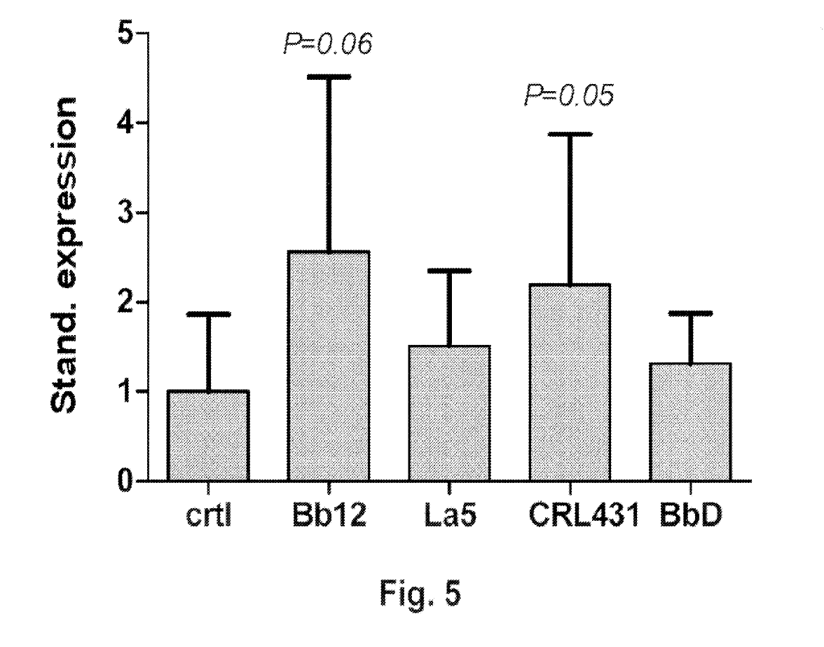

As indicated in FIG. 5, CRL-431 up-regulates ileal APOA4 expression while the three other strains had no significant effect on APOA4 expression.

### Example 5

### Probiotic Strain Down-Regulate SCD-1 Expression in the Skeletal Muscle of Pigs

To investigate whether or not selected probiotic strains regulate skeletal muscle SCD-1 expression in animals, young pigs were fed a standard diet including probiotic bacteria (i.e. Bifidobacterium animalis subsp. lactis strain BB-12® (DSM15954), Lactobacillus acidophilus strain La5 (DSM13241), and Lactobacillus paracasei subsp. paracasei strain CRL431, (ATCC 55544). Pigs fed with the same standard diet but not supplemented with probiotic bacteria served as control. Each group consisted of 8 piglets. At weaning at 4 weeks the animals were moved to pens where they were housed individually and assigned to the corresponding treatments for 14 days. Littermates were assigned to each of the treatments. The number of barrows and gilts in each treatment was the same. The pigs were fed twice daily, receiving an amount of feed corresponding to 4% of their body weight. The probiotics were given on top of the diet every morning.

Permission to Carry Out the Experiment was Granted from the Danish Plant Directorate and the Danish Ministry of Food, Agriculture and Fisheries.

After 14 days of treatment, the pigs were killed and tissues comprising skeletal muscle were sampled and snap-frozen in liquid nitrogen. Gene expression analysis on the distal ileum was performed by quantitative PCR analysis using primers specific for SCD-1. The quantitative PCR analysis was performed essentially as described by Kubista et al. (35).

Primer sequences were,

SCD1-F: 5′- GGGATACAGCTCCCCTCATAG -3′
SCD1-R: 5′- AGTTCCGATGTCTCAAAATGC -3′

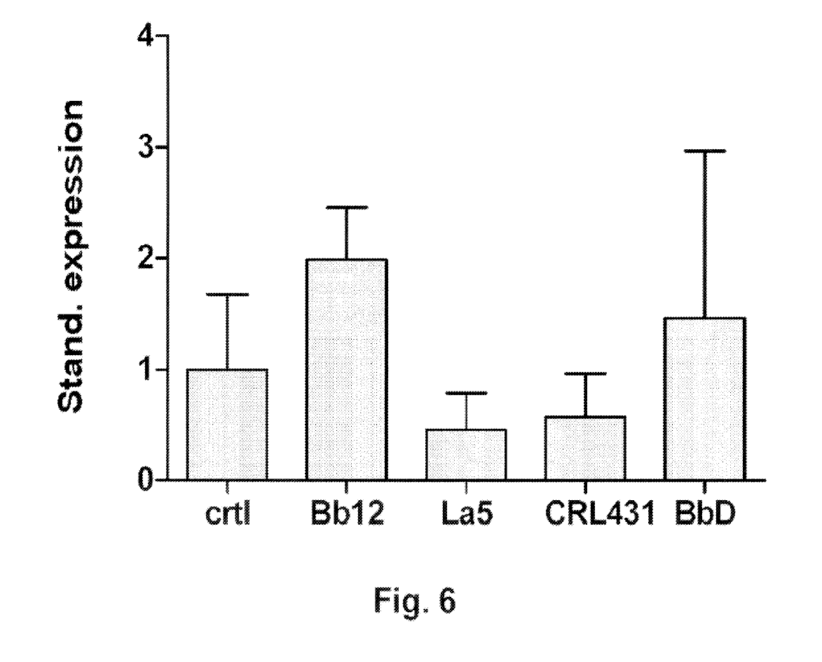

As indicated in FIG. 6, CRL-431 down-regulates skeletal muscle SCD-1 expression by approximately half the level of the non-treated pigs. This is comparable to the down-regulation observed for La-5. In contrast, Bb-12 and BbD (inactivated, dead Bb-12) appears to up-regulate muscle SCD-1 by 100% (for Bb-12) compared to non-treated pigs.

### Example 5

### Effect of CRL431 on Energy Intake, and VAS Scores

The study was an intervention study with 22 healthy men and women, 20 to 45 years old, who would perform three single meal tests of approximately five hours duration. The study had a cross-over design where two different doses of probiotic bacteria or placebo were tested on appetite, ad libitum energy intake, and wellbeing. The participants were randomized to the order of the three different capsules e.g. placebo or 10⁹ CFUL. casei CRL431 or 10¹⁰ CFU L. casei CRL431. At the test days the participants would have either high dose probiotic capsule, low dose probiotic capsule or placebo capsule. These capsules were swallowed at the beginning of a standardized breakfast. Hereafter blood samples were taken and appetite vas registered until serving of an ad libitum lunch. To eliminate carry over effect of the capsules and for the safety of the participants there were a minimum of four weeks between the test days. The evening before the meal test the participants ate a standardized dinner (4.5 MJ) before 8.00 p.m. and were asked to fast hereafter except for a maximum of ½ L of water. On the day of the single meal test, the study participants arrived fasting at the Department at 8.00 a.m. by bus, train, metro or light/slow cycling. The participants were weighed in their underwear before resting and measurement of blood pressure. A venflon catheter was inserted in the right antecubital vein and at time 0 minutes a fasting blood sample was drawn, haemoglobin concentration was determined (>7.5 mmol/l), and appetite was registered on visual analogue scale (VAS) questionnaires before the breakfast (2.5 MJ) and one of the three capsules was served. The participants had a maximum of 14 minutes to eat the breakfast. After the breakfast blood samples were drawn at time: 15, 30, 45, 60, 90, 120, 150, 180, 210 and 240 minutes after beginning of the breakfast adding up to 11 blood samples, 250 ml altogether. Appetite was registered every half hour until ad libitum lunch was served four hours after the breakfast. After the lunch, appetite was registered for the last time and sensory quality of the meal was rated before the participants left the department.

Thirty-one subjects were screened and nine were excluded. A total of 22 subjects (eleven men and eleven women) were randomized and 21 completed all three visits in the study period. Mean age was 27.2 years and mean BMI was 23.6 kg/m2 and did not change significantly during the study period (p>0.1). All participants had blood pressure within a normal range and had normal haemoglobin values, 7.5-10 mmol for women and 8-11 mmol for men, at every visit (Table 1).

TABLE 1
Physical characteristics of participants at baseline.

| | All (n = 21) | Men (n = 11) | Women (n = 10) |
|---|---|---|---|
| Age (y) | 27.2 ± 7.1¹ | 28.0 ± 7.9 | 26.3 ± 6.3 |
| Body composition | | | |
| Weight (kg) | 74.5 ± 12.8 | 84.6 ± 8.1 | 63.3 ± 5.2 |
| BMI (kg/m²) | 23.6 ± 1.4 | 24.2 ± 1.4 | 22.9 ± 1.1 |
| Blood pressure | | | |
| Systolic (mmHg) | 124.8 ± 10.8 | 132.4 ± 7.3 | 116.5 ± 7.4 |
| Diastolic (mmHg) | 78.6 ± 5.1 | 79.5 ± 6.3 | 77.6 ± 3.4 |
| Haemoglobin | 8.8 ± 0.7 | 9.3 ± 0.4 | 8.3 ± 0.6 |
| (mmol/L) | | | |

¹Mean ± SD.

The subjective appetite sensation was expressed as VAS measured in mm. There were no differences in the participants' scores on satiety, fullness, hunger, and prospective food consumption when taking the different capsules adjusted for gender, BMI, and period (p>0.1). In addition, composite appetite which is a sum of hunger, satiety, fullness and prospective intake scores did not differ between the groups (p>0.1). There were no differences between the three kinds of treatments' effect on “desire for something sweet, fatty, salty and savoury” (p>0.1). Furthermore, the participants' well being was rated the same at all three treatments (p>0.1).

The VAS-scores were recalculated to give the area under the curve (AUC). As with the VASscores there were no differences in AUC for any of the VAS questions. However, when looking at the AUC results numerically the participants were most full and satisfied and felt less hunger and thought that they could eat less prospectively when having the capsule with high dose (HD) 10¹⁰ CFU L. casei CRL431 in comparison with placebo and low dose capsules (LD) 10⁹ CFU L. casei CRL431 (Table 2).

TABLE 2
Area under the curve for appetite parameters and well being. All data are presented as
mean ± SD (mm * min) (n = 21).

| | | | Prospective | | | Composite |
|---|---|---|---|---|---|---|
| Treatment | Hunger | Full | food intake | Satiety | Well being | appetite |
| Placebo | 12,794 ± 3,928 | 8,899 ± 4,095 | 13,381 ± 3,935 | 10,646 ± 3,159 | 16,884 ± 4,007 | 10,342 ± 3,644 |
| LD | 12,948 ± 3,798 | 8,634 ± 3,945 | 13,424 ± 3,699 | 10,243 ± 3,545 | 16,577 ± 4,223 | 10,126 ± 3,577 |
| HD | 12,271 ± 3,953 | 9,591 ± 4,036 | 13,231 ± 3,790 | 10,906 ± 3,669 | 16,588 ± 3,974 | 10,749 ± 3,705 |

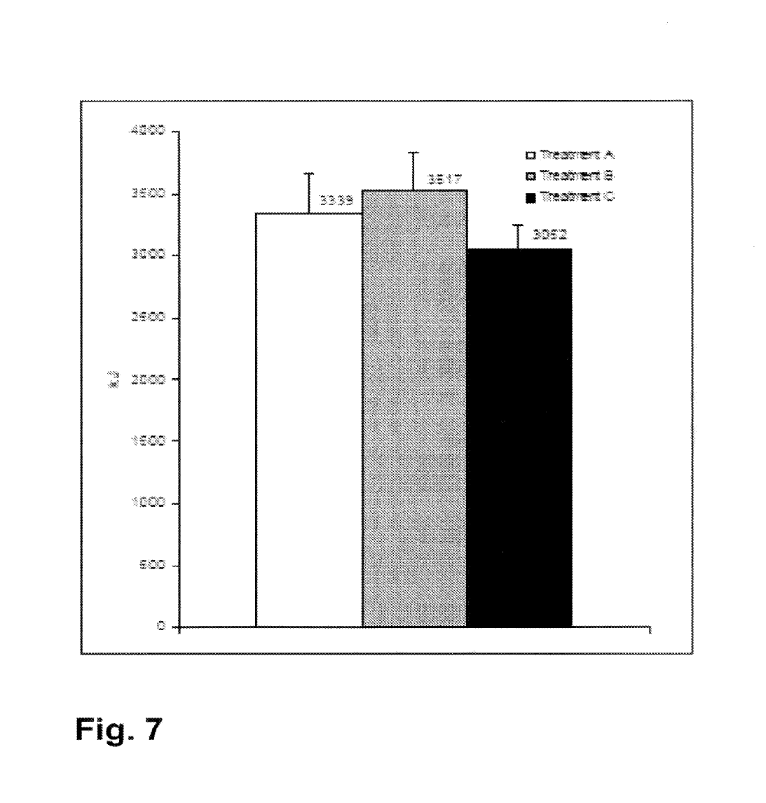

There was an overall effect of treatment on ad libitum energy intake at the lunch meal consumed four hours after taking the different capsules (p=0.04). Posthoc pair wise comparison showed that the energy intake was 455 kJ or approximately 15% lower after having x10 consumed capsule 10¹⁰ CFU L. casei CRL431 in comparison to capsule 10⁹ CFU L. casei CRL431 (p=0.03) (FIG. 7). There was no difference in energy intakes between participants consuming placebo in comparison to capsules with 10¹⁰ CFU L. casei CRL431. These calculations were done after adjusting for gender, BMI, and period. Participants ate less at the ad libitum meal when having 10¹⁰ CFU L. casei CRL431.

### REFERENCE LIST

(1) Hoist J J. The physiology of glucagon-like peptide 1. Physiol Rev 2007; 87(4):1409-39.
(2) Klonoff D C, Buse J B, Nielsen L L et al. Exenatide effects on diabetes, obesity, cardiovascular risk factors and hepatic biomarkers in patients with type 2 diabetes treated for at least 3 years. Curr Med Res Opin 2008; 24(1):275-86.
(3) Verdich C, Flint A, Gutzwiller J P et al. A meta-analysis of the effect of glucagon-like peptide-1 (7-36) amide on ad libitum energy intake in humans. J Clin Endocrinol Metab 2001; 86(9):4382-9.
(4) Tolessa T, Gutniak M, Hoist J J et al. Glucagon-like peptide-1 retards gastric emptying and small bowel transit in the rat: effect mediated through central or enteric nervous mechanisms. Dig Dis Sci 1998; 43(10):2284-90.
(5) Kreymann B, Ghatei M A, Burnet P et al. Characterization of glucagon-like peptide-1-(7-36)amide in the hypothalamus. Brain Res 1989; 502(2):325-31.
(6) Larsen P J, Tang-Christensen M, Hoist J J et al. Distribution of glucagon-like peptide-1 and other preproglucagon-derived peptides in the rat hypothalamus and brainstem. Neuroscience 1997; 77(1):257-70.
(7) Wei Y, Mojsov S. Tissue-specific expression of the human receptor for glucagon-like peptide-I: brain, heart and pancreatic forms have the same deduced amino acid sequences. FEBS Lett 1995; 358(3):219-24.
(8) Shughrue P J, Lane M V, Merchenthaler I. Glucagon-like peptide-1 receptor (GLP1-R) mRNA in the rat hypothalamus. Endocrinology 1996; 137(11):5159-62.
(9) Burrin D G, Petersen Y, Stoll B at al. Glucagon-like peptide 2: a nutrient-responsive gut growth factor. J Nutr 2001; 131(3):709-12.
(10) Tang-Christensen M, Larsen P J, Thulesen J et al. The proglucagon-derived peptide, glucagon-like peptide-2, is a neurotransmitter involved in the regulation of food intake. Nat Med 2000; 6(7):802-7.
(11) Benjamin M A, McKay D M, Yang P C et al. Glucagon-like peptide-2 enhances intestinal epithelial barrier function of both transcellular and paracellular pathways in the mouse. Gut 2000; 47(1):112-9.
(12) Cani P D, Amar J, Iglesias M A et al. Metabolic endotoxemia initiates obesity and insulin resistance. Diabetes 2007; 56:1761-72.
(13) Parlevliet E T, Heijboer A C, Schroder-van der Elst J P et al. Oxyntomodulin ameliorates glucose intolerance in mice fed a high-fat diet. Am J Physiol Endocrinol Metab 2008; 294(1):E142-E147.
(14) Schjoldager B T, Baldissera F G, Mortensen P E et al. Oxyntomodulin: a potential hormone from the distal gut. Pharmacokinetics and effects on gastric acid and insulin secretion in man. Eur J Clin Invest 1988; 18(5):499-503.
(15) Cohen M A, Ellis S M, Le Roux C W et al. Oxyntomodulin suppresses appetite and reduces food intake in humans. J Clin Endocrinol Metab 2003; 88(10):4696-701.
(16) Wynne K, Park A J, Small C J et al. Oxyntomodulin increases energy expenditure in addition to decreasing energy intake in overweight and obese humans: a randomised controlled trial. Int J Obes (Lond) 2006; 30(12):1729-36.
(17) Dakin C L, Small C J, Batterham R L et al. Peripheral oxyntomodulin reduces food intake and body weight gain in rats. Endocrinology 2004; 145(6):2687-95.
(18) Wynne K, Park A J, Small C J et al. Subcutaneous oxyntomodulin reduces body weight in overweight and obese subjects: a double-blind, randomized, controlled trial. Diabetes 2005; 54(8):2390-5.
(19) Wallis K, Walters J R, Forbes A. Review article: glucagon-like peptide 2-current applications and future directions. Ailment Pharmacol Ther 2007; 25(4):365-72.
(20) Tomita R, Igarashi S, Tanjoh K et al. Role of recombinant human glicentin in the normal human jejunum: an in vitro study. Hepatogastroenterology 2005; 52(65):1459-62.
(21) Ayachi S E, Borie F, Magous R et al. Contraction induced by glicentin on smooth muscle cells from the human colon is abolished by exendin (9-39). Neurogastroenterol Motil 2005; 17(2):302-9.
(22) Nygaard R, Nielbo S, Schwartz T W et al. The PP-Fold Solution Structure of Human Polypeptide YY and Human PYY3-36 As Determined by NMRΓçá,Γçí. Biochemistry 2006; 45(27):8350-7.
(23) Murphy K G, Bloom S R. Gut hormones and the regulation of energy homeostasis. Nature 2006; 444(7121):854-9.
(24) Taylor I L. Distribution and release of peptide YY in dog measured by specific radioimmunoassay. Gastroenterology 1985; 88(3):731-7.
(22) Zhang J, Tokui Y, Yamagata K et al. Continuous stimulation of human glucagon-like peptide-1 (7-36) amide in a mouse model (NOD) delays onset of autoimmune type 1 diabetes. Diabetologia 2007; 50(9):1900-9.
(23) Blandino-Rosano M, Perez-Arana G, Mellado-Gil J et al. Anti-proliferative Effect of Pro-inflammatory Cytokines in Cultured Beta Cells is Associated with Erk1/2 Pathway Inhibition: Protective Role of GLP-1. J Mol Endocrinol 2008.
(24) Kubista M, Andrade J M, Bengtsson M et al. The real-time polymerase chain reaction. Mol Aspects Med 2006; 27(2-3):95-125.
(25) Liu C D, Aloia T, Adrian T E et al. Peptide YY: a potential proabsorptive hormone for the treatment of malabsorptive disorders. Am Surg 1996; 62(3):232-6.
(26) Batterham R L, Cowley M A, Small C J et al. Gut hormone PYY (3-36) physiologically inhibits food intake. Nature 2002; 418(6898):650-4.
(27) Kalogeris T J, Rodriguez M D, Tso P. Control of Synthesis and Secretion of Intestinal Apolipoprotein A-IV by Lipid. J Nutr 1997; 127(3):537S.
(28) Shen L, Tso P, Woods S C et al. Hypothalamic Apolipoprotein A-IV Is Regulated by Leptin. Endocrinology 2007; 148(6):2681-9.
(29) Tso P, Liu M. Apolipoprotein A-IV, food intake, and obesity. Physiology & Behavior 2004; 83(4):631-43.
(30) Woods S C. Dietary synergies in appetite control: distal gastrointestinal tract. Obesity (Silver Spring) 2006; 14 Suppl 4:171 S-8S.
(31) Hulver M W, Berggren J R, Carper M J et al. Elevated stearoyl-CoA desaturase-1 expression in skeletal muscle contributes to abnormal fatty acid partitioning in obese humans. Cell Metab 2005; 2(4):251-61.
(32) Zhang J, Tokui Y, Yamagata K et al. Continuous stimulation of human glucagon-like peptide-1 (7-36) amide in a mouse model (NOD) delays onset of autoimmune type 1 diabetes. Diabetologia 2007; 50(9):1900-9.
(33) Blandino-Rosano M, Perez-Arana G, Mellado-Gil J et al. Anti-proliferative Effect of Pro-inflammatory Cytokines in Cultured Beta Cells is Associated with Erk1/2 Pathway Inhibition: Protective Role of GLP-1. J Mol Endocrinol 2008.
(34) Orskov C, Hoist J J, Knuhtsen S et al. Glucagon-like peptides GLP-1 and GLP-2, predicted products of the glucagon gene, are secreted separately from pig small intestine but not pancreas. Endocrinology 1986; 119(4):1467-75.
(35) Kubista M, Andrade J M, Bengtsson M et al. The real-time polymerase chain reaction. Mol Aspects Med 2006; 27(2-3):95-125.
(36) Hansen L, Hartmann B, Bisgaard T et al. Somatostatin restrains the secretion of glucagon-like peptide-1 and -2 from isolated perfused porcine ileum. Am J Physiol Endocrinol Metab 2000; 278(6):E1010-E1018.
(37) Reimer R A, Darimont C, Gremlich S et al. A human cellular model for studying the regulation of glucagon-like peptide-1 secretion. Endocrinology 2001; 142(10):4522-8.
NIH (1998): Blount, A. B. S. et al.: CLINICAL GUIDELINES ON THE IDENTIFICATION, EVALUATION, AND TREATMENT OF OVERWEIGHT AND OBESITY IN ADULTS The Evidence Report. NIH PUBLICATION NO. 98-4083. SEPTEMBER 1998. NATIONAL INSTITUTES OF HEALTH.
Neovius et al (2009) Combined effects of overweight and smoking in late adolescence on subsequent mortality: nationwide cohort study. British Medical Journal) 338, b496.
FAO/WHO (2001) Joint FAO/WHO Expert Consultation on Evaluation of Health and Nutritional Properties of Probiotics in Food Including Powder Milk with Live Lactic Acid Bacteria, October 2001 (ftp://ftp.fao.org/docrep/fao/meeting/009/y6398e.pdf)

1-16. (canceled)

17. A method for reducing body weight in an obese or overweight subject, comprising providing to the subject a composition that consists essentially of Lactobacillus paracasei subsp. paracasei CRL431 strain having accession number ATCC 55544.

18. The method of claim 17, wherein the expression of satiety markers coded by the GCG gene is up-regulated in the intestine of the subject.

19. The method of claim 18, wherein the satiety markers are one or more of Glucagon-Like Peptide-1 (GLP-1), Glucagon-Like Peptide-2 (GLP-2), Oxyntomodulin, Intervening Peptide 2 (IP-2), Glycentin-Related Pancreatic Peptide (GRPP), and Glicentin.

20. The method of claim 19, wherein expression of Apolipoprotein A-IV is up-regulated in the intestine of the subject.

21. The method of claim 19, wherein expression of Peptide YY (PYY) is up-regulated in the intestine of the subject.

22. The method of claim 19, wherein expression of Stearoyl-CoA Desaturase-1 (SCD1) is down-regulated in the skeletal muscles of the subject.

23. The method of claim 19, wherein expression of Apolipoprotein A-IV and PYY are upregulated in the intestine of the subject; and wherein expression of SCD1 is down-regulated in the skeletal muscles of the subject.

24. The method of claim 17, wherein the subject has a Body Mass Index (BMI) of about 25 to about 30.

25. The method of claim 17, wherein the subject has a BMI of about 30 or more.

26. A method for reducing body weight in an obese or overweight subject, comprising providing to the subject a composition that consists essentially of (A) one or more prebiotics and (B) Lactobacillus paracasei subsp. paracasei CRL431 strain having accession number ATCC 55544.

27. The method of claim 26, wherein the prebiotic is one or more of inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides) and polydextrose or a combination thereof.

28. A method for controlling or stabilizing weight gain in a subject in need thereof, comprising providing to the subject a composition that consists essentially of Lactobacillus paracasei subsp. paracasei CRL431 strain having accession number ATCC 55544.

29. The method of claim 28, wherein the expression of satiety markers coded by the GCG gene is up-regulated in the intestine of the subject.

30. The method of claim 29, wherein the satiety markers are one or more of GLP-1, GLP-2, Oxyntomodulin, IP-2, GRPP, or Glicentin.

31. The method of claim 30, wherein expression of Apolipoprotein A-IV is up-regulated in the intestine of the subject.

32. The method of claim 30, wherein expression of PYY is up-regulated in the intestine of the subject.

33. The method of claim 30, wherein expression of SCD1 is down-regulated in the skeletal muscles of the subject.

34. The method of claim 30, wherein expression of Apolipoprotein A-IV and PYY are upregulated in the intestine of the subject; and wherein also expression of SCD1 is down-regulated in the skeletal muscles of the subject.

35. The method of claim 30, wherein the subject has a BMI of about 25 or less.

36. The method of claim 30, wherein the subject has a BMI of about 25 to about 30.

37. The method of claim 30, wherein the subject has a BMI of about 30 or more.

38. A method for controlling or stabilizing weight gain in a subject in need thereof, comprising providing to the subject a composition that consists essentially of (A) one or more prebiotics and (B) Lactobacillus paracasei subsp. paracasei CRL431 strain having accession number ATCC 55544.

39. The method of claim 38, wherein the prebiotic is one or more of inulin, a transgalacto-oligosaccharide, palantinoseoligosaccharide, soybean oligosaccharide, gentiooligosaccharide, oxylooligomers, nondegradable starch, lactosaccharose; lactulose, lactitol, maltitol, FOS (fructo-oligosaccharides), GOS (galacto-oligosaccharides), polydextrose, or a combination thereof.


SEQUENCE LISTING (6 sequences)

SEQ ID NO: 1 - DNA, 24 bp, Artificial Sequence
  Description of Artificial Sequence Synthetic primer
ttcagaatacagaggagaaatcca

SEQ ID NO: 2 - DNA, 22 bp, Artificial Sequence
  Description of Artificial Sequence Synthetic primer
agtcatctgatctggatcatcg

SEQ ID NO: 3 - DNA, 20 bp, Artificial Sequence
  Description of Artificial Sequence Synthetic primer
aaggccaagatcgatcagaa

SEQ ID NO: 4 - DNA, 18 bp, Artificial Sequence
  Description of Artificial Sequence Synthetic primer
gagctcctccgcataggg

SEQ ID NO: 5 - DNA, 21 bp, Artificial Sequence
  Description of Artificial Sequence Synthetic primer
gggatacagctcccctcatag

SEQ ID NO: 6 - DNA, 21 bp, Artificial Sequence
  Description of Artificial Sequence Synthetic primer
agttccgatgtctcaaaatgc